(19) United States
(12) Patent Application Publication
(10) Pub. No.: US 2019/0198150 A1
(43) Pub. Date: Jun. 27, 2019

(54) METHOD FOR TRACKING USER OUTCOMES, SENTIMENT, AND SATISFACTION

(71) Applicant: KangarooHealth, Inc., San Jose, CA (US)

(72) Inventors: Xiaoxu Kang, San Jose, CA (US); Steven Mahana, San Jose, CA (US)

(21) Appl. No.: 16/159,661

(22) Filed: Oct. 13, 2018

Related U.S. Application Data
(60) Provisional application No. 62/572,207, filed on Oct. 13, 2017.

(51) Int. Cl. G16H 20/30; G06F 9/54; G06Q 10/10; G16H 80/00

(52) U.S. Cl. CPC G16H 20/30; G16H 80/00; G06Q 10/1095; G06F 9/542

(57) ABSTRACT

One variation of a method for tracking user satisfaction, recovery, training progress, and outcomes during a recovery and wellness regimen includes: generating a user profile of a user based on user information entered through a user portal; associating the user with a subset of users in a user population based on similarities between the user profile and user profiles of the subset of users; assigning data input types, user check-in schedules, training tasks, and schedules to the user during a recovery and wellness regimen based on historical data from the subset of users; transmitting prompts to the user based on the data input types, user check-in schedules, training plans and schedules; updating the user profile based on responses to the prompts; and notifying a care provider affiliated with the user in response to negative deviation of the user profile from user profiles of patients in the subset of users.

### CROSS-REFERENCE TO RELATED APPLICATIONS

This Application claims the benefit of U.S. Provisional Application No. 62/572,207, filed on 13 Oct. 2017, which is incorporated in its entirety by this reference.

This application is related to U.S. patent application Ser. No. 15/895,981, filed on 13 Feb. 2017, which is incorporated in its entirety by this reference.

### TECHNICAL FIELD

This invention relates generally to the field of patient recovery and more specifically to a new and useful method for detecting user well-being and tracking user progress, outcomes, sentiment, satisfaction and selectively adapting recovery plans to a user's satisfaction and physical states in the fields of patient recovery and training.

### BRIEF DESCRIPTION OF THE FIGURES

FIG. 1 is a flowchart representation of a method; and

FIG. 2 is a flowchart representation of a variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

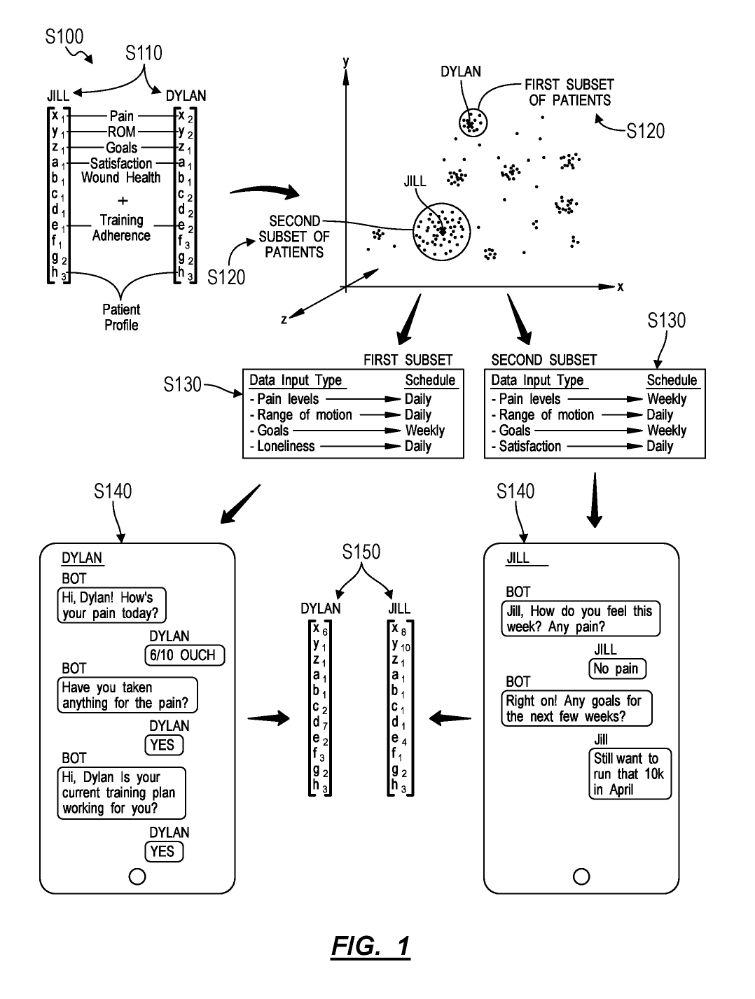

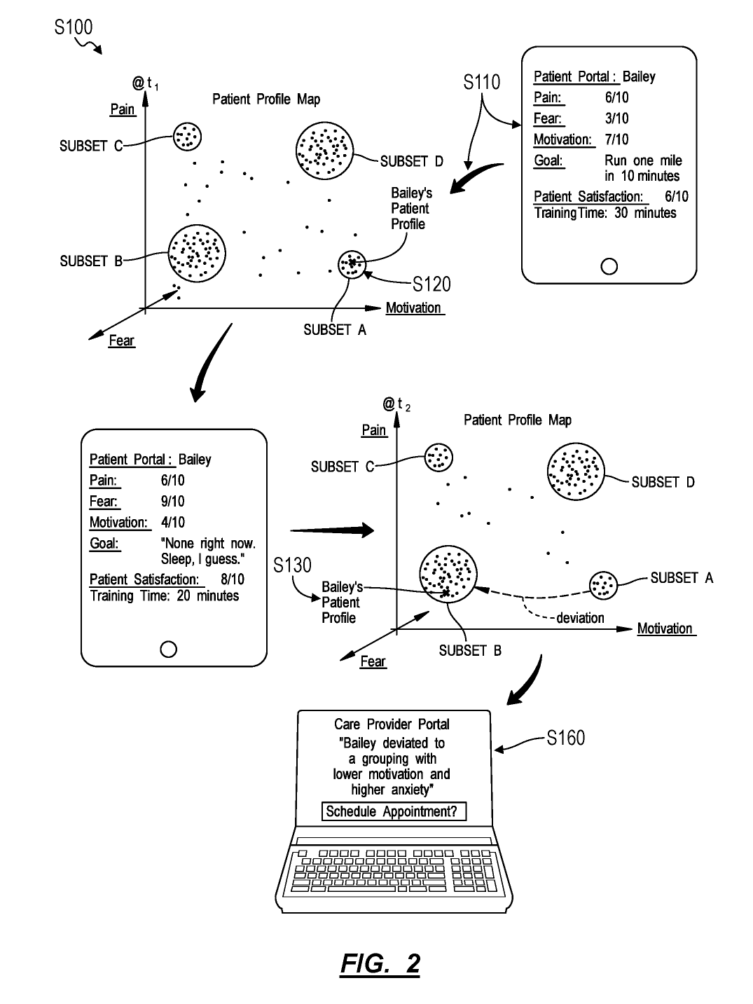

As shown in FIGS. 1 and 2, a method S100 for tracking user satisfaction, recovery, training progress, and outcomes during a recovery and wellness regimen includes: generating a new user profile of a new user based on user information entered through a user portal and user interviews in Block S110; associating the new user with a subset of users in a user population based on similarities between the new user profile and user profiles of patients in the subset of users in Block S120; assigning data input types, user check-in schedules, and training tasks and schedules to the new user during a recovery and wellness regimen based on historical data from the subset of users in Block S130; during recovery and wellness regimen, transmitting prompts to the new user based on the data input types, user check-in schedules, training plans and schedules in Block S140; updating the new user profile based on responses to the prompts in Block S150; and notifying a care provider/trainer in response to negative deviation of the new user profile from user profiles of patients in the subset of users in Block S160.

### 2. Applications

Generally, the method S100 can be executed by a system, such as a computer system or computer network, to: collect patient data before and/or after surgery during a physical therapy regimen; populate a patient profile with patient data; group the patient with past patients with similar known characteristics and outcomes (e.g., range of motion, mobility, pain, wound status, BMI, age, complication history, and/or satisfaction); prompt a care provider (e.g., a doctor, physical therapist, a physical therapy assistant, or other trained specialist) to preemptively assist the patient toward a group with better outcomes if the patient's current group exhibits poor outcomes; and prompt a care provider to preemptively assist the patient if data collected from the patient indicates the patient is deviating from a group with positive outcomes toward a group with less positive outcomes.

For example, the system can execute Blocks of the method S100 in order to: assist a patient enrolled in a physical therapy regimen (e.g., prescribed following a surgery) in setting expectations for her recovery; manipulate frequency and/or types of interactions with the patient in order to motivate the patient to meet or exceed her expectations and in light of predicted changes in the patient's outcome (e.g., satisfaction, pain, etc.) during recovery; and automatically seek care provider assistance for the patient when a negative change in the patient's satisfaction, emotional, and/or physical status is detected. In particular, the system can be configured to: generate and adapt patient profiles; based on a particular patient profile, assign a schedule for prompting a patient to input data of particular data types (e.g., patient satisfaction rating, recovery data, patient information, recovery goals and motivations, and/or general user preferences); serve prompts to a computing device affiliated with the patient according to the schedule; and update the patient profile based on responses to the prompts. Furthermore, the computer system can track changes and deviations in responses to the prompts—and, thus, the patient profile—during the patient's recovery; and serve a notification to a care provider to attend to the patient in response to deviations in the patient profile that appear to indicate a change in the patient's satisfaction (e.g., with the patient's care, recovery expectations, recovery status, etc.), mental status or health of the patient, a change in goals or motivations for recovery, a change in mobility and/or range of motion of an affected area, etc.

By prompting a patient to intermittently input data into a patient portal prior to, during, and after engaging in a physical therapy regimen, the system can function to regularly engage the patient, track the patient's satisfaction with her recovery, respond to physical and psychological changes in the patient's recovery status, and thereby function as a care companion configured to assist the patient in maintaining motivation and treatment adherence throughout recovery and achieving her recovery goals. For example, the system can serve relevant and timely prompts to the patient based on recorded patient preferences stored in the patient's profile, past responses to similar prompts, and/or preferences of other patients exhibiting characteristics similar to those of the patient.

Furthermore, the system can regularly query the patient for her personal expectations and goals for recovery (e.g., pain levels, mobility outcome, etc.) following a surgery, selectively prompt a care provider to intervene in the patient's recovery in light of patient responses that indicate low or diminishing patient satisfaction with her progress toward these goals, and to maintain (or increase) the patient's satisfaction with her recovery throughout the recovery process with regular patient check-ins. Thus, the system can function to assist patients in avoiding dissatisfaction, disappointment, discouragement, and/or depression that may arise from failure to meet or exceed personal expectations by dynamically adjusting frequency, types of check-ins, training regimens with the patient and/or selectively prompting a care provider to engage the patient when such engagement may be most effective in elevating the patient's short- and long-term satisfaction. For example, a first patient may exhibit a low pain tolerance and low motivation to recovery from a surgical operation while a second patient may exhibit a higher pain tolerance and may be highly motivated to recover from similar surgeries. The system can thus assign: a relaxed and slower physical therapy regimen with a high tolerance for deviations from the physical therapy regimen and frequent psychological check-ins to the first patient; and a more aggressive, faster-paced physical therapy regimen with a low tolerance for deviations from the physical therapy regimen and less frequent psychological check-ins to the second patient. Therefore, the first patient may avoid discouragement due to failure to meet milestones of the more aggressive, faster-paced physical therapy regimen of the second patient, and the second patient may avoid boredom or frustration resulting from repeatedly exceeding expectations of the relaxed and slower physical therapy regimen of the first patient. As the first patient begins to exhibit motivation more like that of the second patient (or other motivated past patients), the system can automatically transition the first patient to a more aggressive, faster-paced physical therapy regimen with lower tolerance for deviations from the physical therapy regimen and less frequent psychological check-ins. Similarly, as the second patient begins to exhibit motivation more like that of the first patient (or other less-motivated past patients), the system can automatically transition the second patient to a less aggressive, slower-paced physical therapy regimen with higher tolerance for deviations from the physical therapy regimen and more frequent psychological check-ins; the system can also prompt a physical therapist, doctor, a specialist, and/or a nurse to manually check-in with the second patient, since the second patient may be deviating toward a lower-satisfaction outcome.

In particular, the system can maintain historical patient data, including demographic, surgery types, pain levels, mobility characteristics, joint range of motion, wound recovery characteristics, and patient satisfaction, etc. over time pre-surgery, during recovery, and post-recovery for a population of past patients. For a new patient or patient in the midst of recovery, the system can: group this patient with a subset of past patients in the patient population based on similarities in patient demographic, surgery type, pain levels, etc. between this patient and the group; and predict the patient's outcome (e.g., final mobility, final wound recovery characteristics, final pain level, final satisfaction with the surgery, etc.) based on outcomes of past patients in the group. Given a positive predicted outcome for the patient, the system can implement goals, apply automated check-in frequency and type, apply automated training frequency and type, and prompt care provider check-ins, etc.—similar to those of the group of past patients—for this new patient. Similarly, given a negative or less-positive predicted outcome for the patient, the system can automatically implement more aggressive automated check-ins, and/or prompt more frequent care provider check-ins, etc. in order to move the patient out of this group of past patients. Similarly, in response to detecting deviation from one group with positive outcomes to another group with less-positive outcomes, the system can automatically prompt a care provider to intervene in the patient's recovery in order to preempt low patient satisfaction and physical status pre-recovery, during or post-recovery.

Therefore, by leveraging historical patient and patient outcome data to customize automated and manual communications with a current patient based on the patient's actual physical and/or emotional status over time, the system can achieve higher patient satisfaction throughout her recovery and upon completion of her recovery while also efficiently allocating human resources (i.e., doctor and/or physical therapist time and energy) to patients most in need of human intervention.

Blocks of the method S100 can be implemented in conjunction with a physical therapy program prescribed to a patient following a surgical operation, such as: a hip replacement surgery; knee replacement surgery; shoulder arthroscopy surgery; carpal tunnel release surgery; an anterior cruciate ligament (“ACL”) reconstruction surgery; a lumbar spinal fusion surgery; etc. Furthermore, Blocks of the method can be implemented in conjunction with a physical therapy program prescribed to a patient: following an injury; as a course of treatment and/or rehabilitation from a chronic disease or ailment (e.g., strokes, amyotrophic lateral sclerosis, multiple sclerosis, heart disease, cardiac abnormalities and arrhythmias, epilepsy, Parkinson's disease, etc.); as a course of treatment for isolated pain in a joint or other musculoskeletal component of the patient's body; as a course of treatment for limited mobility of a joint or other musculoskeletal component of the patient's body; etc.

The method and techniques described herein are directed toward patient rehabilitation during physical therapy following a surgical operation. However, Blocks of the method S100 can also be implemented in conjunction with healing, recovery, overall wellness tracking, and/or any other training regimen of a patient, athlete, and/or any other type of user. Furthermore, Blocks of the method S100 can be implemented to track short-term recovery and healing (e.g., between two and six months after a surgery or injury) and well as mid-term (e.g., over six months to two years) and long-term (e.g., over two to five years) health, wellness, and/or training.

### 3. User Interface

Blocks of the method are described below as executed by a system, such as a mobile computing device, a cloud-based computer, a mainframe computer system, a grid-computer system, a computer network, or any other suitable computer system, such as in the form of a local computing device or a remote server.

In one implementation, the system can execute Blocks of the method S100 remotely at a remote computer system (e.g., a remote computer network or server) and transmit prompts to a computer system or a computing device (e.g., a mobile computing device such as a smartphone, tablet computer, a wearable device, an intelligent personal assistant, etc.) affiliated with or assigned to the patient. In this implementation, the computing device affiliated with the patient can (locally) execute a native recovery application accessible by the patient and including a user portal (or “patient portal) into which the patient may enter responses to inquiries generated by the remote computer system.

Alternatively, the system can execute Blocks of the method S100 locally at the computer system affiliated with the patient in an in-browser application or a recovery native application executing on the computer system. Thus, the computer system can render the patient portal within the in-browser application or native recovery application.

In the foregoing implementations, the system can serve to the patient portal: physical therapy modules; queries prompting a patient to input pictures and/or videos documenting the patient's recovery process, range of motion, mobility, wound health, etc.; and/or surveys, inquiries, and/or other chat prompts inquiring about the patient's mental and physical well-being during the physical therapy regimen.

Therefore, the patient portal can include a chat portal (or a “chat bot”) with which the patient may communicate orally or through text input. In one implementation, the chat bot can serve dynamic prompts, queries, and/or surveys customized to a patient's personality, patient profile, and/or interface preferences as described below. For example, the chat bot can generate and serve prompts that mimic human linguistic patterns, dialects, and characteristics familiar or similar to linguistic patterns, dialects, and characteristics of the patient to which the chat bot served the prompts. Thus, the chat bot can mimic a human personality to engage a patient in customized and dynamic conversations throughout the physical therapy regimen, such that the chat bot can function as a recovery companion to the patient and can seek assistance for the patient from a care provider when the patient appears to be struggling—emotionally and/or physically—during the physical therapy regimen.

For example, the chat bot can receive patient information for a particular patient indicating the particular patient is from the Midwest, enjoys swimming and cycling, recently tore her right rotator cuff partially, and has commenced a physical therapy regimen to rehabilitate her right rotator cuff so that she may swim the English Channel in eight months. Thus, the chat bot can intermittently serve: prompts to the particular patient inquiring about the progress of her swimming training while mimicking the particular patient's Midwestern dialect; prompts inquiring how the particular patient's shoulder feels while swimming and cycling; hyperlinks to articles about recent English Channel attempts; suggestions for local cycling routes; suggestions for physical therapy exercises helpful for preventing common injuries for swimmers and cyclists; motivational quotes; etc.

Additionally, the system can cooperate with computing devices (e.g., a personal computer, workstation, computer network, and/or mobile computing device) affiliated with care providers and executing a care provider portal accessible by care providers, such as doctors, nurses, technicians, and/or physical therapists. Thus, the system can cooperate with computing devices affiliated with care providers to receive patient medical information (e.g., medical history), recovery plans and schedules, etc.

As described below, the system can serve reminders, alerts, and/or prompts to the care provider portal notifying the care provider of patient goals, patient mental and physical status, when a particular patient requires additional assistance, and/or deviations in a patient's profile during the patient's recovery.

However, the system can serve prompts to the patient profile and/or the care provider portal in any other suitable way.

### 5. Patient Profiles

As shown in FIGS. 1 and 2, Block S110 of the method S100 recites generating a patient profile of a patient based on patient information entered through a user portal in Block S110. Generally, the system can receive patient information entered through the patient portal and/or care provider portal and generate a patient profile based on the patient information entered.

In particular, the system can serve prompts to the patient portal at various stages prior to, throughout, and after completion of a physical therapy regimen to prompt a patient (and/or a care provider) to enter data describing the patient's physical status (e.g., level of pain, range of motion, and/or general mobility), mental status (e.g., level of anxiety, motivations or goals, and/or confidence in efficacy of treatment), and current satisfaction with care. The system can serve prompts as a series of sequential questions through the chat bot, as a generalized survey with predefined responses, and/or as an open-ended survey with comment input regions in which a patient may enter qualitative responses. Based on responses to the prompts, the system can generate the patient profile summarizing the patient's satisfaction, mental and physical status of the patient, and describing key factors which may correlate to efficacy and outcomes of particular physical therapy regimens and schedules; level of companionship and external motivation from a care provider and/or the chat bot that the patient may require during the physical therapy regimen; etc.

In one implementation, prior to commencement of the physical therapy regimen (e.g., prior to a surgical operation or during an intake visit to a physical therapist or physician), the system can serve prompts to the patient portal (and/or to the care provider portal) to query a patient to enter information relevant to gauging: the patient's pre-operative understanding of risks and rewards of the surgery and expectations for post-operative recovery and physical therapy; and the patient's demographic information, medical history, and mental health status, which may affect the outcome of the surgery and efficacy of the post-operative physical therapy regimen. For example, the system can serve prompts to the patient portal to query a patient to enter information, such as: an assessment of her current pain levels (e.g., rating pain on a scale of “Zero:: no pain” to “Ten: extremely painful”); perceived pain tolerance (e.g., “no tolerance for pain,” “little tolerance for pain,” “moderate tolerance for pain,” or “hardly affected by pain”); fear and/or anxiety regarding post-operative pain; fear and/or anxiety regarding losing range of motion after surgery; demographic information, such as age, weight, height, gender, ethnicity, and pre-operative activity level; medical history (e.g., prior surgeries and/or injuries, pre-existing conditions, allergies, and/or prior recovery timelines); level or degree of understanding of the upcoming surgery, recovery timeline, and physical therapy regimen; perceived risk of depression; etc. Thus, the system can generate a pre-operative patient profile detailing a patient's physical and emotional preparation for a surgery and populate the patient profile with patient information and patient expectations for the surgery and post-operative recovery.

Furthermore, in another implementation, during a time window immediately succeeding a surgical operation and prior to commencement of a physical therapy regimen with a therapist (e.g., within two to three weeks after a surgery), the system can intermittently (e.g., hourly, daily, and/or weekly) serve prompts to the patient portal querying a patient to enter information pertaining to the patient's understanding of the efficacy of the surgery and expectations for the imminent physical therapy regimen. For example, the system can serve prompts to the patient portal to query a patient to enter information, such as: an assessment of her current pain levels; range of motion assessment metrics for regions of the patient's body affected by the surgery; anxiety levels regarding post-operative pain, loss of mobility, loss of range of motion, and/or physical therapy; and level of understanding of her physical therapy regimen and timeline for recovery. Furthermore, the system can access and/or prompt the patient and/or care provider to enter post-operative images, scans, and/or pictures of a surgical wound (i.e., to track wound healing and notify a care provider to intervene in response to a complication with the wound, such as an infection in the wound. (Alternatively, the system can prompt the patient to input a qualitative assessment of wound health and healing.) Thus, the system can generate a post-operative patient profile detailing a patient's physical and emotional status after a surgery and prior to commencement of the physical therapy regimen with a therapist; and update the patient profile to reflect changes in the patient's expectations for outcome of the surgery and imminent physical therapy.

In another implementation, after commencing a physical therapy regimen and during the physical therapy regimen, the system can intermittently serve prompts to the patient portal to enter information related to the patient's current pain and anxiety levels, understanding of recovery milestones, perceived efficacy of the operation and the prescribed physical therapy regimen, physical changes in range of motion and mobility, etc. For example, the system can query a patient to enter information, such as: an assessment of her current pain levels; comfort level with her current pain levels; anxiety regarding permanent or temporary loss of mobility, loss of range of motion; anxiety about continued physical therapy, treatment, and/or recurrence of injury; wound health (e.g. through a qualitative assessment input by the patient to the patient portal and/or by uploading a picture of the wound); etc. Additionally, the system can query the patient to enter metrics related to efficacy of the patient's physical therapy regimen, such as: current range of motion; quantity and frequency of pain medication ingested; current mobility; current activity level; etc. Thus, the system can update the patient profile to detail a patient's physical status and emotional well-being during physical therapy and reflect changes in the patient's expectations and anxiety during recovery.

In the foregoing implementation, the system can serve prompts according to patient check-in schedules assigned to the patient, as described below. For example, the system can serve prompts daily at 6:15 am when a patient typically wakes and at 7:45 pm when the patient typically completes her daily physical therapy exercises. Furthermore, the system can serve prompts every Tuesday and Thursday at 4:15 pm after the patient's biweekly visit to her physical therapist.

Furthermore, upon completion of the physical therapy regimen (e.g., several weeks, months, or years after a last visit with a physical therapist; after restoration of range of motion or reduction of pain levels; and/or achievement of prescribed physical therapy milestones), the system can intermittently (e.g., biweekly, monthly, semi-annually, or yearly) serve prompts to the patient portal after completion of the physical therapy regimen to enter information related to the patient's on-going range of motion, pain, mobility, and other implications and/or complications (e.g., wound infections) after surgery and physical therapy. Thus, the system can maintain and update the patient profile after physical therapy and surgery to monitor outcomes of a physical therapy regimen (and surgery) and track complications that may arise over time.

However, the system can query the patient for information pertaining to the patient's physical and/or emotional health prior to, during, and after a physical therapy regimen and update the patient's patient profile accordingly in any other way.

### 5.2 Recovery Goals & Satisfaction

Prior to a surgery, during recovery from surgery, and/or after completion of a physical therapy regimen, the system can serve prompts to the patient portal inquiring about the patient's motivations for surgery and goals for recovery. Generally, throughout recovery from surgery, a patient's goals and motivations may change. Thus, the system can monitor changes in goals and motivations and, in response to deviations, deduce a patient's level of comfort, happiness, and/or optimism about the outcome of the surgery and suitability of the physical therapy regimen.

In particular, the system can serve prompts—to the patient portal—inquiring about the patient's post-surgery goals. For example, the patient may wish to walk without pain, cycle across Europe, compete in swimming competitions, and/or play with his children or grandchildren. Additionally, the patient may indicate he is training for an event in ten weeks or wishes to walk at his grandson's high school graduation. Thus, the system can record the patient's motivations for surgery, goals for recovery, and desired timeline for recovery in the patient's profile. Intermittently (e.g., daily, weekly, monthly, etc.) throughout the patient's recovery, the system can prompt the patient to enter his motivations and goals into the patient portal, and the system can verify alignment between the patient's current motivations and goals and his past motivations and goals. The system can therefore track whether a patient's motivations have changed and, in response to such a change in patient motivation, query the patient for why the patient's motivations or goals have changed (e.g., due to depression, loneliness, or dissatisfaction with progress during recovery). The system can additionally or alternatively remind the patient of his prior goals in order to maintain the patient's motivation for recovery.

For example, prior to an ACL repair surgery, the system can serve a prompt to the patient portal such as “After your surgery, what would you like to do that you are unable to do currently?” The patient may enter a response such as “Run a marathon within a year of surgery.” Later (e.g., post-surgery), the system can again serve the prompt “After you recover from surgery, what would you like to do that you are unable to do currently?” to which the patient may enter a response such as “nothing” or “to be determined.” The system can: qualify or quantify this change in the patient's response; and notify a care provider, such as a psychologist or psychiatrist, to assist the patient if such a change is common to a group of past patients who exhibited low or negative post-recovery satisfaction or if this change indicates that this patient is beginning to exhibit characteristics similar to those of past patients with low or negative post-recovery satisfaction, as described below.

However, the system can collect and track a patient's motivations for surgery and/or goals for recovery by any other means.

### 5.2 Tracking Patient Satisfaction

In one implementation, throughout recovery, the system can prompt a patient to input a patient satisfaction rating evaluating the patient's current satisfaction with level of care provided by a care provider, current satisfaction with her recovery progress, current satisfaction with the efficacy of her physical therapy regimen, etc. For example, immediately after a surgery, the system can: prompt a patient to enter her overall satisfaction with her recovery; record an overall satisfaction rating of 3/5; and prompt the patient to explain reasons contributing to the overall satisfaction rating, to which the patient may enter “My physical therapist does not appear to care about my pain levels,” “I do not feel like I am making any progress,” and/or “My physical therapy exercises are too difficult.” The system can then record explanations for the overall satisfaction ratings and serve notifications to relevant parties, such as a physical therapist or doctor, informing them of the patient's satisfaction. Furthermore, as described below, the system can suggest changes to improve the patient's satisfaction, such as other care providers which may meet the patient's expectations.

Additionally or alternatively, the system can calculate a patient's satisfaction level based on responses (or a subset of responses) to other prompts input by the patient to the patient portal. In one implementation, the system can calculate the patient's satisfaction level as a linear combination (e.g., average) of a subset of responses to prompts in the patient portal. For example, the system can: prompt a patient rate her care provider's bedside manner, difficulty of her physical therapy exercises, and rate her overall level of happiness; record the patient's responses rating her care provider's bedside manner (e.g., “4 out of 10”), difficulty of her physical therapy exercises (e.g., “9 out of 10”), and rate her overall level of happiness (e.g., “4 out of 10”); and extract an overall satisfaction rating based on a linear combination of the patient's responses (e.g., “5.667 of 10”). In another implementation, the system can extract the patient's level of satisfaction from another metric known to correlate with the patient's level of satisfaction. For example, the system can determine that patients' rating of their care provider's bedside manner strongly correlates with their overall satisfaction with recovery. Thus, in response to recording a patient's rating of her care provider's bedside manner of “9/10”, the system can extract a high overall patient level of satisfaction.

However, the system can track the patient's satisfaction level intermittently throughout the patient's recovery in any other suitable way.

### 5.3 Patient Vector

The system can then aggregate responses to prompts served to the patient portal prior to surgery and throughout the patient's recovery to define the patient profile as a vector of elements with each element of the vector representing a category of responses to prompts served to the patient portal. For example, a first element of the vector can describe the patient's pain tolerance; a second element of the vector can describe the patient's current anxiety level; a third element of the vector can describe a difference between the patients expected timeline for recovery and a projected timeline for recovery prescribed by a care provider; a fourth element of the vector can describe the patient's current satisfaction with care provided by a care provider; etc.

In one implementation, the system can populate a vector of predefined dimensions with patient data input into the patient portal by the patient and/or care provider. For example, the system can populate an n-dimensional vector in which each element of the vector represents a data input type (or category) of patient data, such as: pain level, anxiety level, range of motion, confidence in care provider care, confidence in efficacy of treatment, and overall mood, etc. With each prompt served to the patient portal, the system can request information to fill each element or a subset of elements in the patient-specific vector. For example, at a first time, the system can serve prompts inquiring about a patient's satisfaction with care, pain level, anxiety level, range of motion and record the patient's responses as quantitative values in corresponding elements in the patient's assigned vector. At a second time, the system can serve prompts inquiring about the patient's pain level, satisfaction, overall mood, and confidence in efficacy of treatment and again record the patient's responses as quantitative values in corresponding elements in the patient's assigned vector.

In the foregoing example, in response to receipt of new patient responses to such queries (i.e., as the system receives patient information and responses to such prompts), the system can amend or replace values within the patient's assigned vector to correspond with current relevant information input by the patient. Alternatively, the system can generate a new vector in response to each set of responses input to the patient portal.

In another implementation, the system can calculate values of the vector as a linear combination (e.g., mean, median, or weighted average) of values previously entered into the patient portal. For example, at a first time, a patient may input a pain level of “3 out of 5;” at a second time, the patient may input a pain level of “3 out of 5”; and at a third time, the patient may input a pain level of “4 out of 5.” Thus, the system can calculate an average pain level of “3.33333” and populate an “average pain level” element of the vector with the value “3.333333.” Additionally, at a fourth time, the patient may input a pain level of “2 out of 5” and the system can update the “average pain level” element of the vector with “3.”

Alternatively, the system can populate a vector of dynamic dimensions. In particular, in response to receiving a first set of patient data input (i.e., prior to an operation), the system can populate a vector with quantitative values describing each item of available patient data (e.g., a six-by-one dimension vector). In response to receipt of subsequent patient data (i.e., as the system queries the patient for additional information, such as daily during the patient's post-surgery recovery), the system can append the vector with additional quantitative values representing these additional patient data. For example, at a first post-operative time, the patient may input current pain level, current range of motion, and current anxiety level; and the system can append a vector assigned to the patient (and currently containing pre-operative patient data) with new quantitative elements representing the patient's pain levels (e.g., “6/10”), current range of motion (e.g., “25 degrees”), and current anxiety levels (e.g., “3/10”) at the first post-operative time. At a second, later post-operative time, the patient may input into the patient portal current pain level, current anxiety level, and a target recovery goal. The system can then append the patient's assigned vector with new quantitative elements representing the patient's target recovery goal (e.g., “eight weeks to 90% mobility”), current pain level (e.g., “4/10”), and current anxiety level (e.g., “6/10”) at the second post-operative time.

However, the system can collect patient data of any other type and in any other way over time and can store these patient data in any other format or data structure.

### 6. Group

Block S120 of the method S100 recites associating the patient with a subset of patients in a patient population based on similarities between the patient vector and patient vectors of patients in the subset of patients. Generally, the system can compare a patient vector of a particular patient to other patient vectors of prior patients and affiliate the particular patient with a group of similar patients with similar patient vectors to the patient vector of the particular patient.

In one implementation, shown in FIG. 1, the system compiles vectors representing current patients (or unknown outcomes) and past patients (of known outcomes) into a multidimensional map of patient data describing various patient characteristics and outcomes. From the multidimensional map, the system can extract groups (e.g., clusters) of similar patient vectors. For example, the system can implement clustering techniques, such as K-means clustering, nearest neighbor, latent Dirichlet allocation, and/or nonlinear kernels supplied to linear methods, etc. to identify distinct cohorts of near patient vectors (or “points” representing unique patients) within the multidimensional patient map. In this implementation, each distinct cohort of patient vectors can thus represent similar patients exhibiting similar: past, current, and/or post-operative pain, anxiety, goals, motivations, mobility, joint range of motion, and/or satisfaction; demographic values; surgery type; injury; etc. and engaged in a physical therapy program managed by the system.

As the system collects additional patient data (e.g., pain, satisfaction, mobility, motivation, etc.) over time, such as during recovery periods and post recovery, the system can repeat this process to update the patient map and to recalculate groups of patients exhibiting similarities along various dimensions. As described below, the system can therefore predict an outcome for a current patient based on proximity of the patient to a group of past patients and the outcomes (e.g., common or average outcomes) of these past patients. Furthermore, the computer system can also compare cohorts of patients across a sequence of patient maps to identify a positional change of a particular point—representing a particular patient currently engaged in a physical therapy regimen—that may indicate that the patient is now exhibiting characteristics more similar to past patients who experienced low intra-recovery satisfaction, low post-recovery satisfaction, high risk of recidivism, high risk of re-injury, poor post-recovery mobility, high post-recovery pain, etc.

In a similar implementation, the system can compare a patient vector—generated from data collected over a first duration following a surgery—to other patient vectors generated during similar periods of time, similar surgeries of past patients (or following commencement of a physical therapy regimens for past patients). For example, the system can compare a patient vector generated from data collected from this patient over a two-week period post surgery with other patient vectors generated from data collected over similar two-weeks post-surgery periods. Thus, the system can compare patient vectors at similar stages of recovery in order to group a current patient with past patients of known outcomes.

Alternatively, the system can implement the foregoing methods and techniques to group patient vectors representing data collected over indiscriminate periods of time, such as starting with first pre-operative query responses, terminating at the current time for current patients, and terminating at the conclusion of physical therapy regimens and or extending well past conclusion of physical therapy regimens for past patients.

The system can also prioritize particular elements and/or data input types and deprioritize other data input types during comparison between patient vectors. In particular, the system can identify commonalities among a first subset of elements within a set of patient vectors (e.g., similar or identifying values within elements of the patient vectors) and dissimilarities among a second subset of elements within the set of patient vectors. However, the system can prioritize the first subset of elements higher than the second subset of elements and, thus, define a group corresponding to the set of patient vectors. Alternatively, the system can prioritize the second subset of elements higher than the first subset of elements and, thus, reject the group.

For example, the system can: prioritize a patient satisfaction rating over other elements of the patient vector and (exclusively) compare a particular patient's satisfaction rating element of the patient vector to patient satisfaction rating elements of other patient vectors. Therefore, for example, the system can group patients with high satisfaction ratings with other patients with high satisfaction ratings. Thus, as described below, the system can project patient satisfaction ratings throughout her recovery.

In one variation of the method S100, the system can extract groups of patient vectors, label the group as a cluster, and, for each patient vector within the cluster, calculate a confidence score describing how well each patient vector fits within the cluster.

However, the system can identify groups and/or clusters of similar patient vectors in any other suitable way.

### 7. Therapy Protocols

The system can then assign a physical therapy regimen (or program) and data input requirements to the particular patient based on historical data from the subset of patients with which a particular patient is grouped (as described above). Generally, the system can assign to a particular patient a physical therapy regimen that was executed (or deemed effective) for patients similar to the particular patient such as with guidance from a physical therapist or doctor. For example, the custom recovery plan can include a set of physical therapy exercises, a schedule for performing the set of physical therapy exercises, and range of motion milestones for determining efficacy of the set of physical therapy exercises. The system can then serve videos of the set of physical therapy exercises to the patient portal to assist the patient in performing the physical therapy exercises at home or otherwise without a physical therapist present.

Based on the patient vector, the system can also extract information about care providers who worked well with similar patients in the past and can, thus, recommend a particular physical therapist or doctor. For example, a patient may feel more comfortable working with a physical therapist with interests similar to those of the patient; thus, the system can recommend a care provider with similar interests to the patient.

### 7.1 Data Collection Schedule

Block S130 of the method S100 recites assigning data input types and patient check-in schedules to the patient during a physical therapy regimen based on historical data from the subset of patients in Block S130. Generally, the system can assign data input requirements to a patient, such as a schedule for when to serve prompts for the patient to input patient data and data input types corresponding to elements of the patient vector, based on data input requirements of other patients similar to (i.e., in the same group as) the patient. In particular, the system can generate a patient check-in schedule for serving prompts to a patient portal and prompting a patient to input patient data that the system can implement to update the patient vector and/or adjust the physical therapy regimen.

In one implementation, the system can detect times of day when a patient may be likely to respond to prompts and define general times of day for issuing intermittent prompts in the patient check-in schedule. The system can define the patient check-in schedule for a patient based on effective or preferred patient check-in schedules of patients similar to the patient characteristics of the patient represented in the patient vector. For example, the system can determine that patients in a group of similar patients may describe themselves as night owls and generally prefer to respond to prompts just prior to falling asleep (e.g., at 11:45 pm). After detecting a patient vector that falls within the group, the system can assign a patient check-in schedule for serving prompts to a patient portal affiliated with the patient around 11:45 pm or when the patient indicates she is preparing to go to bed.

Alternatively, the system can serve a set of test prompts to the patient portal at various times of day, times of week, times of month, etc. to align a schedule for serving prompts with regular times when a patient is likely to input complete responses into the patient portal. For example, a patient may be most receptive to prompts from the system in the morning when she wakes, over lunch, and when she feels alone and desires companionship. Thus, the system can generate a schedule for regularly serving prompts in the morning around her usual wakeup time (e.g., 7 am), over lunch (e.g., 12:15 pm), and on days when she feels alone (e.g., Saturday and Sunday).

Additionally or alternatively, the system can align frequency of patient prompts with a patient's desire for companionship and personal accountability for complying with the physical therapy regimen as deduced from a patient vector (e.g., from proximity of the patient vector to patient vectors of past patients of known satisfaction, loneliness, motivation, etc. as similar stages of recovery or post-recovery). For example, the system can determine that a current patient exhibits independence and consistent motivation during recovery based on alignment of the patient vector of the current patient with patient vectors of a group of past patients who exhibited independence and consistent motivation during recovery. Thus. the system can define the patient check-in schedule to issue prompts infrequently (e.g., every other day) during the patient's physical therapy regimen to avoid overloading the patient with information and inquiries. However, the system can determine a second patient vector aligns with other patients who exhibited a high-degree emotional dependence on the chat bot for companionship during recovery (e.g., an elderly patient who lives alone). Thus, the system can define the patient check-in schedule to issue prompts frequently (e.g., up to six times a day).

The system can also define a patient check-in schedule (and/or assign a predefined patient check-in schedule) for the current patient based on the phase of a patient's recovery. In particular, as described above, the system can vary the frequency of prompts among each phase. In one implementation, the system can issue prompts to the patient portal during the following recovery phases: prior to surgery, immediately after a surgery and prior to commencement of physical therapy, during physical therapy, and after completion of physical therapy. For example, the system can: serve a singular prompt regarding patient anxiety and motivation a few hours prior to a scheduled surgery; serve a series of eight prompts regarding patient pain and medication consumption over a period of four weeks immediately after a surgery and prior to commencement of physical therapy; serve prompts regarding patient satisfaction, pain, and motivation multiple times per day (e.g., three times per day) during a first week of a physical therapy regimen and gradually taper these prompts to once per day toward an end of the physical therapy regimen; and/or serve prompts for patient satisfaction biweekly for three months, monthly for the following three months, and bimonthly for the following six months after completion of the physical therapy regimen; etc.

The system can therefore adjust type and frequency of prompts served to the patient as a function of time since surgery and/or progress through an assigned physical therapy regimen.

### 7.2 Expectations & Recovery Schedule

As described above, intermittently during recovery from a surgery or during physical therapy, the system can serve prompts inquiring about a patient's goals and preferences. Based on the patient's goals and recovery schedules of patients similar to the patient, the system can assign a recovery schedule to the patient defining key range of motion and strength milestones, frequency of exercise and physical therapy visits, and a tolerance threshold for deviations from the recovery schedule (e.g., number of missed sessions, number of setbacks, or failure to meet particular milestones).

In particular, the system can align patient expectations, such as represented in the patient vector, with a recovery schedule extracted from prior recovery schedules of similar patients. The system can define the recovery schedule to define key milestones for recovery (e.g., restoration of 50% of full range of motion within a month of surgery, 75% of full range of motion with no pain at three months, full range of motion within six months of surgery, and full range of motion with no pain at eight months from surgery.) The system can also define frequency of visits to a physical therapist and/or physician and performance of prescribed physical therapy exercises (e.g., multiple times a day, daily, or five times a week).

Additionally or alternatively, the system can define a tolerance threshold for deviations from the recovery plan. In one implementation, the system can define the tolerance threshold to depend upon a patient's motivations or goals for surgery. For example, for a patient with aggressive goals following surgery, the system can assign a low tolerance threshold for deviations from the plan. Alternatively, the system can define the tolerance threshold based on tolerance of pain and/or perceived patient persistence extracted from a patient's patient vector. For example, for patients with aggressive goals, high pain tolerance, and/or history of expedited recovery from surgery, the system can assign to the patients aggressive and fast-paced recovery schedules with limited tolerance for deviations from the schedule (e.g., maximum three missed sessions during recovery). However, patients with low pain tolerance, less aggressive goals, and/or history of deviating from physical therapy regimens, the system can assign to the patients slow-paced recovery schedules with a wide tolerance for deviations (e.g., maximum of ten missed sessions during recovery).

### 8. Prompts During Recovery

As shown in FIGS. 1 and 2, Blocks S140 and S160 of the method S100 recite during the physical therapy regimen, transmitting prompts to the patient based on the data input types and patient check-in schedules in Block S140; and notifying a care provider in response to deviation of the patient vector from patient vectors of patients in the subset of patients in Block S160. Generally, the system can execute Blocks S140 and S160 to serve prompts inquiring about data input types and according to the patient check-in schedules and training regimens assigned to the patient in Block S130; based on responses to prompts input to the patient portal, update the patient vector accordingly; and in response to deviations from the patient's prior group, the system can selectively notify a care provider to assist the patient.

In particular, as described above, the system can, through a chat bot, serve customized inquiries, reminders, and alerts according to the patient's patient check-in schedule in order to update and track changes in the patient's profile over time. Thus, the system can function to check-in with patients and monitor changes in the patient's mental and physical health to record both physical and emotional side-effects (or complications) of the patient's customized physical therapy regimen.

In one implementation, the system can intermittently serve reminders to a patient of past goals or motivations, update the patient on milestones she has achieved, and provide an estimated time until a patient has achieved her goals for recovery. Similarly, the system can notify the patient of deviations from the plan, inquire whether the deviations were intentional or inadvertent, and, if deliberate, serve a series of prompts to gauge how the system can modify data input types and schedules and the physical therapy regimen to avoid future deviations. Thus, the system can function to both adapt to variable patient motivations and goals and motivate the patient to stick to previously-cited plans and hold the patient accountable.

### 9. Adaptation

As shown in FIGS. 1 and 2, Block S150 recites updating the patient vector based on responses to the prompts in Block S150. Generally, during a physical therapy regimen, the system can update the patient vector with responses to prompts input by a patient to the patient portal.

As described above, the system can update the patient vector in response to each response (or a subset of responses) to prompts served to the patient portal. Thus, the system can track changes in each data category (i.e., element of the patient vector) during recovery from surgery. Then, in response to updating the patient vector, the system can again map the vector to the multidimensional map; and identify a group of patient vectors similar to the updated patient vector. The system can then check whether the patient vector remains in a consistent group over time or deviates from an initial group. In one implementation, during recovery, the system can: calculate a degree (i.e., magnitude or distance) of deviation and/or a rate of deviation (i.e., magnitude of deviation over time) of the patient vector from a first time to a second time. In response to the degree of deviation exceeding a threshold degree of deviation predefined by the system, the rate of deviation exceeding a threshold rate of deviation, and/or in response to detecting the patient vector shifting away from a group of past patient vectors exhibiting positive outcomes (e.g., low pain levels, expedited recovery timelines, restoration of range of motion, and/or high patient satisfaction ratings); intermittently serve automated prompts and/or queries to a patient portal associated with the current patient to preempt further deviation from the group toward a group exhibiting less-positive (or negative) outcomes (e.g., high pain levels, slow recovery timelines, incomplete restoration of range of motion, and/or low patient satisfaction ratings); and/or selectively serve prompts to a care provider portal associated with a care provider of the current patient to intervene with the patient's physical therapy regimen in order to preempt further deviation from the group toward a group exhibiting less-positive (or negative) outcomes.

For example, the system can: detect deviation of a patient vector of a current patient away from a first group of past patients exhibiting relatively positive outcomes (e.g., low anxiety, high patient satisfaction, and/or high range of motion); modify the patient check-in schedule to increase frequency of prompts served to the patient portal; and/or increase frequency of automated queries served to the patient portal regarding particular data input types, such as patient satisfaction, pain, anxiety, wound health, range of motion, motivation, etc. Therefore, the system can preempt deviation of the patient vector from the first group to a second group of past patients exhibiting less-positive (or negative outcomes). Similarly, in another example, the system can detect deviation of a patient vector of a current patient away from a first group of past patients exhibiting relatively positive outcomes (e.g., low anxiety, high patient satisfaction, and/or high range of motion) to a second group of past patients exhibiting less-positive outcomes (e.g., high anxiety, low patient satisfaction, high incidence of opioid addiction, and/or high pain levels) due to a change in the current patient's physical well-being (e.g., adversely affected by illness, an additional injury, and/or pain) and/or change in the current patient's mental health (e.g., increased symptoms of depression, dependence on opioids and/or prescription narcotics, and/or increase anxiety or fear about loss of mobility). Then the system can: selectively serve prompts to a care provider portal to manually intervene with the patient's physical therapy regimen, such as by scheduling a check-up appointment; modify the patient check-in schedule to increase frequency of prompts served to the patient portal; and/or increase frequency of automated queries served to the patient portal regarding particular data input types, such as patient satisfaction, pain, anxiety, range of motion, motivation, etc. Therefore, the system can prompt a care provider to preempt deviation of the patient vector from the first group to the second group.

In another example, the system can detect a patient vector of a current patient remains within a first group of past patients exhibiting relatively positive outcomes (e.g., low anxiety, high patient satisfaction, low incidence of opioid addiction, and/or high range of motion) and/or shifts to a second group of similar positive or more-positive outcomes. In this example, the system can maintain the patient check-in schedule and data input types assigned to the current patient; and/or prompt a patient to continue her physical therapy regimen.

### 9.1 Patient Satisfaction

As shown in FIGS. 1 and 2, in a similar variation, the system can adapt a patient check-in schedule, training regimens, and/or data input types to changes in a patient's satisfaction rating in order to increase the patient's confidence in her recovery, care provider(s), and/or progress. For example, based on a patient's group (as described above), the system can generate a patient satisfaction model from which the system can predict the patient's satisfaction ratings throughout her physical therapy regimen. In response to detecting a decrease of the patient's satisfaction rating—thereby deviating from the patient satisfaction model (and, thus, deviation from the patient's group)—after a month into her physical therapy regimen, the system can increase frequency of prompts (i.e., modifying the patient check-in schedule), prompt the patient to enter an explanation for the decrease in the patient's satisfaction (e.g., dissatisfaction with a care provider's bedside manner, loneliness, physical therapy regimen is too aggressive etc.), suggest alternative care provider(s) for the patient, and/or modify the physical therapy regimen to conform with the patient's expectations and abilities.

However, the system can track patient satisfaction and adapt physical therapy regimens and patient check-in schedules to improve patient satisfaction in any other suitable way.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for managing, communicating, and adapting expectations during a physical therapy plan comprising:
generating a first patient profile of a first patient based on patient information entered through a user portal;
associating the first patient with a subset of patients in a patient population based on similarities between the first patient profile and patient profiles of patients in the subset of patients;
assigning data input types and data input schedules to the first patient during a physical therapy plan based on historical data from the subset of patients;
during the physical therapy plan, transmitting prompts to the first patient based on the data input types and data input schedules;
updating the first patient profile based on responses to the prompts; and
notifying a care provider affiliated with the user in response to deviation of the first patient profile from patient profiles of patients in the subset of patients.

2. A method for tracking user satisfaction, recovery, training progress, and outcomes during a recovery and wellness regimen comprising:
generating a new user profile of a new user based on user information entered through a user portal;
associating the new user with a subset of users in a user population based on similarities between the new user profile and user profiles of patients in the subset of users;
assigning data input types, user check-in schedules, and training tasks and schedules to the new user during a recovery and wellness regimen based on historical data from the subset of users;
during recovery and wellness regimen, transmitting prompts to the new user based on the data input types, user check-in schedules, training plans and schedules;
updating the new user profile based on responses to the prompts; and
notifying a care provider affiliated with the user in response to negative deviation of the new user profile from user profiles of patients in the subset of users.
